(19) United States
(12) Patent Application Publication
(10) Pub. No.: US 2023/0144756 A1
(43) Pub. Date: May 11, 2023

(54) MULTI-SENSOR INTERFEROMETRY SYSTEMS AND METHODS

(71) Applicant: ABIOMED, Inc., Danvers, MA (US)

(72) Inventor: Alexander Ship, Danvers, MA (US)

(73) Assignee: ABIOMED, Inc., Danvers, MA (US)

(21) Appl. No.: 17/984,652

(22) Filed: Nov. 10, 2022

Related U.S. Application Data
(60) Provisional application No. 63/278,320, filed on Nov. 11, 2021.

(51) Int. Cl. A61M 60/13; A61M 60/531; A61M 60/414; A61M 60/226; A61M 60/888; G02B 6/42

(52) U.S. Cl. CPC A61M 60/13; A61M 60/226; A61M 60/414; A61M 60/531; A61M 60/888; G02B 6/4215; G02B 6/4298

(57) ABSTRACT

A ventricular support system including a light source, a fiber optic splitter, two or more filters, an intravascular blood pump having two or more sensor heads, and a photodetector is disclosed. At least some of the light transmitted by the light source is split by the fiber optic splitter such that a portion is transmitted to each of the two or more filters. Each portion of light is filtered by one of the two or more filters and transmitted to one of the two or more sensor heads. Light beams reflected from each of the two or more sensor heads are combined by the fiber optic splitter. At least some of the combined light beams are received by the photodetector. By only using a single light source and a single photodetector with the two or more sensor heads, the ventricular support system may have improved portability.

### CROSS REFERENCE TO RELATED APPLICATION

This application claims the benefit of U.S. Provisional Application No. 63/278,320, which was filed on Nov. 11, 2021 and is incorporated by reference herein.

### TECHNICAL FIELD

The present technology relates to systems and methods for providing hemodynamic support to a patient with an intravascular blood pump. In some implementations, the blood pump includes a plurality of optical pressure sensors for measuring a plurality of pressures within the patient’s vascular system.

### BACKGROUND

Intravascular blood pumps may be used to provide hemodynamic support to the heart of a patient (e.g., during a high-risk percutaneous coronary intervention). For example, an intravascular blood pump may be introduced percutaneously into a patient’s blood vessel (e.g., the femoral artery) and guided through the patient’s vascular system in order to support or replace the pumping action in the patient’s heart. An intravascular blood pump may include an inlet area, an outlet area, a cannula, a motor housing, a catheter, and one or more sensors. During operation, an intravascular blood pump may be positioned such that the cannula extends through an opened cardiac valve to enable blood to be pumped through the cardiac valve. For example, blood may be drawn into one or more openings of the inlet area, channeled through the cannula, and expelled through one or more openings of the outlet area by a motor disposed within the motor housing. The one or more sensors may, for example, be used to measure one or more pressures within one or more chambers of the patient’s heart. Such measurements may, for example, be used to monitor the patient and/or assist with the positioning of the blood pump within the patient’s vascular system. Signals from the one or more sensors may be provided to a controller through one or more lines (e.g., optical and/or electrical lines) extending through the blood pump. To improve the portability of these types of systems, there is a need to reduce the size and/or power consumption of the controller and/or the intravascular blood pump.

### BRIEF SUMMARY

Systems and methods for providing hemodynamic support to a patient with an intravascular blood pump are disclosed. In some implementations, a ventricular support system includes a light source, a fiber optic splitter, two or more filters, an intravascular blood pump having two or more sensor heads, and a photodetector. At least some of the light transmitted by the light source is split by the fiber optic splitter such that a portion is transmitted to each of the two or more filters. Each portion of light is filtered by one of the two or more filters and delivered to one of the two or more sensor heads. Light beams reflected from each of the two or more sensor heads are combined by the fiber optic splitter. At least some of the combined light beams are received by the photodetector. By only using a single light source and a single photodetector with the two or more sensor heads, the ventricular support system may have improved portability.

One aspect of the present disclosure relates to a ventricular support system that includes a light source, an intravascular blood pump, a first filter, a second filter, a first fiber optic splitter, and a first photodetector. The light source is configured to transmit light. The intravascular blood pump includes a first sensor head and a second sensor head, wherein the first sensor head is positioned distally relative to the second sensor head. The first filter is directly or indirectly coupled to the first sensor head. The first filter permits a first sub-spectrum of the light transmitted by the light source to pass through it. The second filter is directly or indirectly coupled to the second sensor head. The second filter permits a second sub-spectrum of the light transmitted by the light source to pass through it, wherein the first and second sub-spectrums are different. The first fiber optic splitter is directly or indirectly coupled to the first and second filters. The first fiber optic splitter is configured to split at least some of the light transmitted by the light source such that a first portion is transmitted to the first filter and a second portion is transmitted to the second filter. The first fiber optic splitter is also configured to combine a plurality of light beams reflected from the first and second sensor heads. The first photodetector is configured to receive at least some of the combined plurality of light beams reflected from the first and second sensor heads.

In some implementations, the first filter is directly coupled to the first sensor, and the second filter is directly coupled to the second sensor. In some implementations, the first fiber optic splitter is (a) coupled to the first filter through a first optical fiber extending through at least a portion of a catheter of the intravascular blood pump, and (b) coupled to the second filter through a second optical fiber extending through at least a portion of the catheter. In some implementations, the first filter is coupled to the first sensor head through a first optical fiber extending through at least a portion of a catheter of the intravascular blood pump, and the second filter is coupled to the second sensor head through a second optical fiber extending through at least a portion of the catheter. In some implementations, the first filter is a short-pass filter, and the second filter is a long pass filter. In some implementations, the first and second filters are band-pass filters. In some implementations, the first and second sub-spectrums overlap. In some implementations, the first and second sub-spectrums do not overlap.

In some implementations, the system further includes a beam splitter coupled to the first fiber optic splitter through a first optical fiber extending through at least a portion of a catheter of the intravascular blood pump. In some implementations, each of the first and second sensor heads comprise a cavity and a pressure-sensitive membrane. In some implementations, the cavity and the pressure-sensitive membrane of each of the first and second sensor heads form part of a Fabry-Perot cavity. In some implementations, the system further includes a mirror configured to reflect at least some of the light transmitted by the light source towards the first photodetector, and each of the first and second sensor heads comprises a mirror attached to a bottom surface of the pressure-sensitive membrane that faces the cavity.

In some implementations, the system further includes a third filter directly or indirectly coupled to a third sensor head of the intravascular blood pump, wherein the third filter permits a third sub-spectrum of the light transmitted by the light source to pass through it, and wherein the first, second, and third sub-spectrums are different, wherein the first fiber optic splitter is configured to split at least some of the light transmitted by the light source such that a third portion is transmitted to the third filter, and wherein the first fiber optic splitter is configured to combine a plurality of light beams reflected from the first, second, and third sensor heads.

In some implementations, the system further includes a controller communicatively coupled to the light source and the first photodetector. In some implementations, the first photodetector is configured to capture an image of a combined interference pattern formed by the combined plurality of light beams reflected from the first and second sensor heads, and the controller is configured to digitally filter the image to separate the interference patterns created by the first and second sensor heads.

In some implementations, the system further includes (a) a second photodetector configured to receive at least some of the combined plurality of light beams reflected from the first and second sensor heads, (b) a third filter directly or indirectly coupled to the first photodetector, wherein the third filter permits the first sub-spectrum of the light transmitted by the light source to pass through it, (c) a fourth filter directly or indirectly coupled to the second photodetector, wherein the fourth filter permits the second sub-spectrum of the light transmitted by the light source to pass through it, and (d) a second fiber optic splitter directly or indirectly coupled to the third and fourth filters, wherein the second fiber optic splitter is configured to split at least some of the combined plurality of light beams reflected from the first and second sensor heads such that a first portion is transmitted to the third filter and a second portion is transmitted to the fourth filter.

Another aspect of the present disclosure relates to a method that includes (a) transmitting, from a light source, a first light beam, (b) splitting, with a fiber optic splitter, at least some of the first light beam received by the fiber optic splitter into a second light beam and a third light beam, (c) filtering, with a first filter, at least some of the second light beam received by the first filter, wherein the first filter permits a first sub-spectrum of the second light beam to pass through it, (d) filtering, with a second filter, at least some of the third light beam received by the second filter, wherein the second filter permits a second sub-spectrum of the third light beam to pass through it, and wherein the first and second sub-spectrums are different, (e) reflecting, with a first sensor head of an intravascular blood pump, at least some of the filtered second light beam to produce a first reflected light beam, (f) reflecting, with a second sensor head of the intravascular blood pump, at least some of the filtered third light beam to produce a second reflected light beam, (g) combining, with the fiber optic splitter, the first and second reflected light beams, and (h) receiving, with a photodetector, at least some of the combined first and second reflected light beams.

In some implementations, the method further includes deriving a ventricular pressure and an aortic pressure from the at least some of the combined first and second reflected light beams received by the photodetector. In some implementations, each of the first and second sensor heads comprises a cavity and a pressure-sensitive membrane. In some implementations, the cavity and the pressure-sensitive membrane of each of the first and second sensor heads form part of a Fabry-Perot cavity.

### BRIEF DESCRIPTION OF DRAWINGS

FIG. 1 illustrates a ventricular support system that includes an intravascular blood pump positioned within the heart of a patient.

FIG. 2 illustrates aspects of an optical pressure sensor of the intravascular blood pump of FIG. 1 in greater detail.

FIG. 3 illustrates aspects of the intravascular blood pump of FIG. 1 in greater detail.

FIGS. 4A and 4B illustrate aspects of an optical pressure sensor of the intravascular blood pump of FIG. 1 in greater detail.

FIGS. 5A and 5B illustrate aspects of an optical pressure sensor of the intravascular blood pump of FIG. 1 in greater detail.

FIG. 6 illustrates a multi-sensor interferometry system.

FIG. 7 illustrates a multi-sensor interferometry system.

FIG. 8 illustrates a multi-sensor interferometry system.

### DETAILED DESCRIPTION

Implementations of the present disclosure are described in detail with reference to the drawing figures wherein like reference numerals identify similar or identical elements. It is to be understood that the disclosed implementations are merely examples of the disclosure, which may be embodied in various forms. Well-known functions or constructions are not described in detail to avoid obscuring the present disclosure in unnecessary detail. Therefore, specific structural and functional details disclosed herein are not to be interpreted as limiting, but merely as a basis for the claims and as a representative basis for teaching one skilled in the art to variously employ the present disclosure in virtually any appropriately detailed structure.

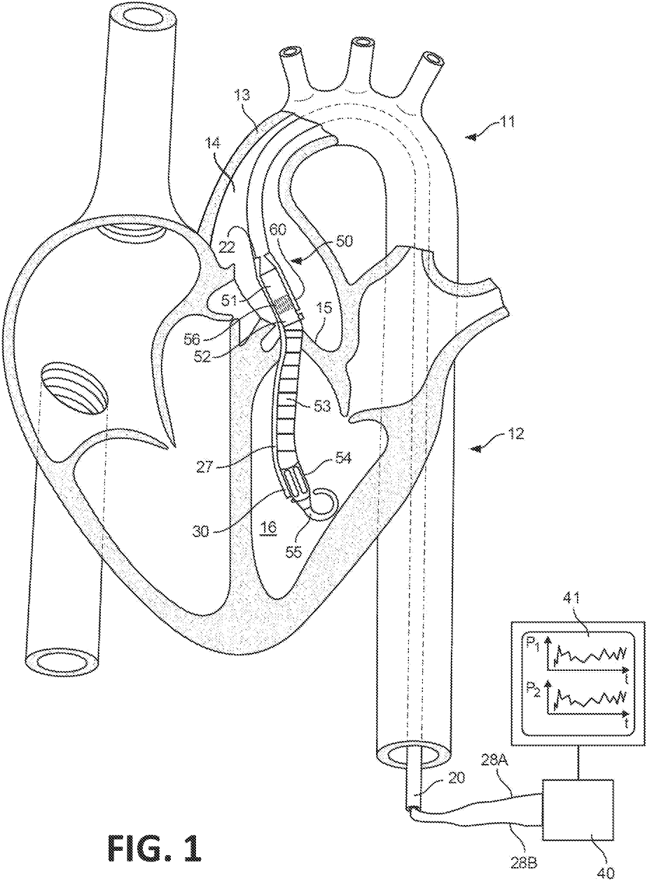

FIG. 1 illustrates a ventricular support system that includes an intravascular blood pump 50 and a controller 40 (e.g., an Automated Impella Controller® from Abiomed, Inc., Danvers, MA). Blood pump 50 includes a catheter 20, a motor section 51, a pump section 52, a cannula 53, an inlet area 54, a soft-flexible tip 55, an outlet area 56, and optical sensor heads 30 and 60. Optical fibers 28A and 28B extend through catheter 20. Controller 40 may transmit and/or receive signals from sensor heads 30 and 60 through optical fibers 28A and 28B, respectively. Tip 55 may be configured, for example, as a “pigtail” or in a J-shape to assist with stabilizing blood pump 50 in the heart of a patient. During operation, blood may be drawn into one or more openings of inlet area 54, channeled through cannula 53, and expelled through one or more openings of outlet area 56 by a motor (not shown) disposed in motor section 51. In some implementations, the blood flow inlet and outlet areas may be reversed, such that during operation, blood may be drawn into one or more openings of outlet area 56, channeled through cannula 53, and expelled through one or more openings of inlet area 54.

As shown in FIG. 1, blood pump 50 may be positioned in a patient’s heart. For example, blood pump 50 may be inserted percutaneously via the femoral artery (not shown) into the aorta 11, across the aortic valve 15, and into the left ventricle 16. The aorta 11 includes the descending aorta 12, the aortic arch 13, and the ascending aorta 14. During operation, blood pump 50 may entrain blood from the left ventricle 16 and expel blood into the ascending aorta 14. As a result, blood pump 50 performs at least some of the work normally done by the patient’s heart. The hemodynamic effects of blood pump 50 may include, for example, an increase in cardiac output, improvement in coronary blood flow resulting in a decrease in left ventricular end-diastolic pressure (LVEDP), pulmonary capillary wedge pressure (PCWP), myocardial workload, and oxygen consumption. In other implementations, blood pump 50 may, for example, be inserted percutaneously via the axillary artery (not shown) into the aorta 11, across the aortic valve 15, and into the left ventricle 16. In other implementations, blood pump 50 may, for example, be inserted directly into the aorta 11, across the aortic valve 15, and into the left ventricle 16. In other implementations, blood pump 50 may be positioned within the right side of the patient’s heart and support right-sided circulation.

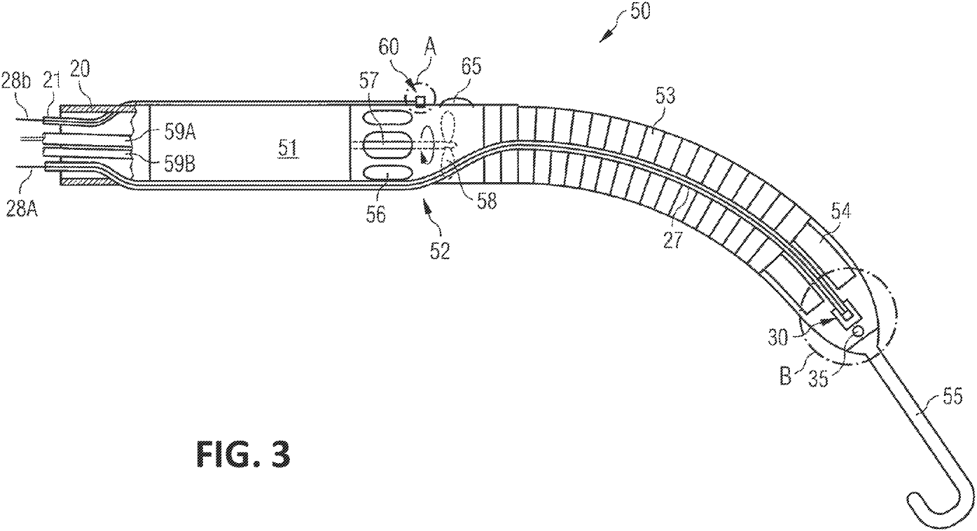

Controller 40 monitors and controls blood pump 50. For example, as noted above, controller 40 may transmit and/or receive signals from sensor heads 30 and 60 through optical fibers 28A and 28B, respectively. Controller 40 may also transmit and/or receive additional signals. For example, controller 40 may monitor and control the power (e.g., current and/or voltage) provided to the motor (not shown) disposed in motor section 51 through one or more power-supply lines extending through catheter 20 (e.g., power-supply line 59A of FIG. 3). As another example, controller 40 may monitor and control a pressure and/or flow rate of a purge fluid delivered to the motor (not shown) disposed in motor section 51 through one or more purge-fluid lines extending through catheter 20 (e.g., purge-fluid line 59B of FIG. 3) to prevent blood from entering the motor. In some implementations, the purge fluid is a dextrose solution (e.g., 5% dextrose in water with 25 or 50 IU/mL of heparin). Data derived by controller 40 from these signals may be displayed on a display 41. The data derived by controller 40 may be used by a clinician to monitor a patient and/or adjust the positioning of blood pump 50 within the patient. In some implementations, controller 40 is connected to an external power source (e.g., a battery or an electrical outlet of a power grid). In some implementations, controller 130 comprises an internal power source (e.g., a battery). When electrical power is supplied by means of a battery, a patient may be afforded a greater degree of mobility.

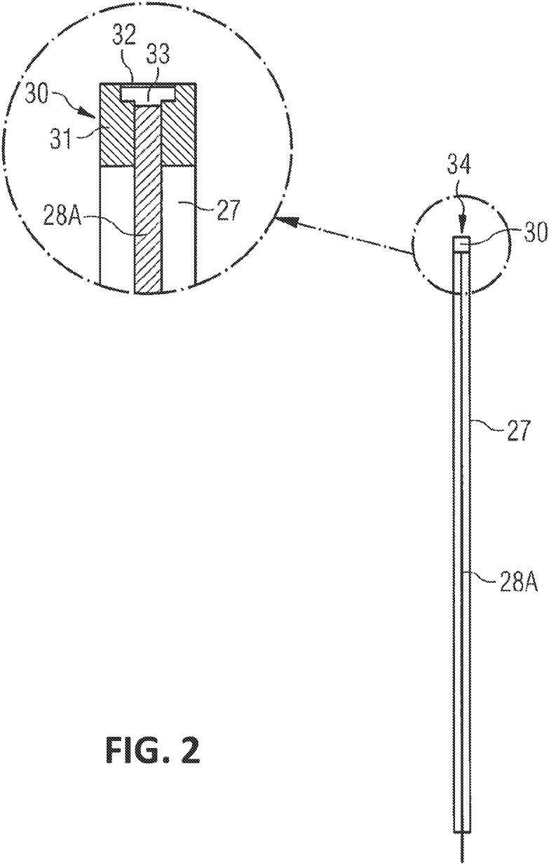

Signals from optical sensor heads 30 and 60 may be used by controller 40 to measure ventricular pressure and aortic pressure, respectively. Signals from sensor heads 30 and 60 may also be used by controller 40 to measure the flow of blood through blood pump 50 (e.g., by evaluating the difference between the ventricular and aortic pressures), measure contractility (e.g., the inherent ability of the heart muscle to contract), and/or detect bending of tip 55. In some implementations, sensor heads 30 and 60 may be micro-electro-mechanical systems (MEMS). Sensor heads 30 and 60 may include a pressure-sensitive membrane separated from the tips of the distal ends of optical fibers 28A and 28B, respectively, by a cavity (see, e.g., membrane 32 and cavity 33 of FIG. 2). In some implementations, these components may form a Fabry-Perot cavity. In some implementations, the membrane is a glass membrane (e.g., SiO2) or ceramic membrane (e.g., Si₃N₄). In some implementations, the membrane has an additional coating (e.g., a silicone coating) on a surface facing the surroundings. In other implementations, the membrane has no additional coating on its surface facing the surroundings.

As shown in FIG. 1, sensor heads 30 and 60 are fixed externally on cannula 53 and pump section 52, respectively. In some implementations, sensor heads 30 and/or 60 are positioned within a depression provided in the external surface of blood pump 50. The depression may protect sensor heads 30 and/or 60 from colliding with, for example, a sluice valve or a hemostatic valve when blood pump 50 is being introduced into the patient’s vascular system. In some implementations, sensor heads 30 and/or 60 are positioned by a bulge projecting beyond the periphery of blood pump 50. Much like the depression provided in some implementations, the bulge may protect sensor heads 30 and 60 from colliding with, for example, a sluice valve or a hemostatic valve when blood pump 50 is being introduced into the patient’s vascular system. In some implementations, the bulge has a U-shape or an O-shape. In some implementations, the bulge may be formed by a bead of bonding agent. In some implementations, the bulge may be welded on or soldered onto the external surface of blood pump 50. In some implementations, the bulge may form an integral part of blood pump 50.

FIG. 2 illustrates aspects of sensor head 30 in greater detail. However, the following description of sensor head 30 and its associated components is equally applicable to sensor head 60 and its associated components. As shown in FIG. 2, optical fiber 28A extends through a tube 27. Sensor head 30 is located at the distal end 34 of tube 27. In some implementations, one or more additional optical fibers may extend through tube 27. As shown in FIG. 1, tube 27 exits catheter 20 at point 22 and is attached to the external surface of blood pump 50 along motor section 51, pump section 52, and cannula 53. In some implementations, tube 27 may encase all or some of the portion of fiber 28A that extends through catheter 20. In some implementations, tube 27 may terminate at or near point 22 (e.g., within 5 cm of point 22). In some implementations, tube 27 may be formed from a polymer (e.g., polyurethane) and/or a metal alloy (e.g., nitinol).

Sensor head 30 includes a housing 31, which contains a pressure-sensitive membrane 32 separated from the tip of the distal end of optical fiber 28A by a cavity 33. In some implementations, membrane 32 is a glass membrane (e.g., SiO2) or a ceramic membrane (e.g., Si₃N₄). As shown, a top surface of membrane 32 is exposed to the surrounding environment, and a bottom surface of membrane 32 is exposed to cavity 33. In other implementations, a coating (e.g., a silicone coating) may be disposed on the top surface of membrane 32. During operation, membrane 32 is deformed in dependence on the size of a pressure acting on sensor head 30. In some implementations, membrane 32 may be aligned orthogonally to a longitudinal axis of blood pump 50 to reduce the noise generated by the operation of blood pump 50.

In some implementations, the tip of the distal end of optical fiber 28A, membrane 32, and cavity 33 may form a Fabry-Perot cavity. In such implementations, a first partially reflective mirror may be disposed on the tip of the distal end of optical fiber 28A, and a second partially reflective mirror may be attached to the bottom surface membrane 32. During operation, controller 40 may transmit a light beam to sensor head 30 through optical fiber 28A. As the light beam interacts with the Fabry-Perot cavity, it is partially and multiply reflected by the first and second partially reflective mirrors to produce a plurality of interfering rays. For example, as the light beam contacts the first partially reflective mirror disposed on the tip of the distal end of optical fiber 28A, it is partially reflected. Furthermore, the portion of the light signal that travels through the first partially reflective mirror is then partially reflected by the second partially reflective mirror attached to the bottom surface of membrane 32. The plurality of reflected, interfering rays travel back through optical fiber 28A and may be received by, for example, a photodetector (e.g., a charge coupled device (CCD) or complementary metal oxide semiconductor (CMOS) image sensor) disposed in controller 40. As membrane 32 moves in response to the pressure acting on sensor head 30, the interference pattern formed by the plurality of reflected, interfering rays changes. Controller 40 may, for example, derive pressure measurements (e.g., ventricular pressure and/or aortic pressure) from these changing interference patterns.

In other implementations, the Fabry-Perot cavity may be formed differently. For example, a glass substrate may be disposed between the tip of the distal end of optical fiber 28A and cavity 33. In such implementations, the distal end of optical fiber 28A may be attached to the glass substate with an adhesive, and the first partially reflective mirror may instead be disposed on a surface of the glass substrate that faces cavity 33. In other implementations, sensor head 30 may be incorporated into a different type of interferometer. For example, rather than being incorporated into a Fabry-Perot interferometer, sensor head 30 may be incorporated into a Michelson interferometer or a Mach-Zehnder interferometer. In such implementations, sensor head 30 may, for example, include a single mirror attached to the bottom surface membrane 32, rather than a pair of partially reflective mirrors, as described above.

FIG. 3 illustrates aspects of blood pump 50 in greater detail. As shown, a drive shaft 57 protrudes from motor section 51 into pump section 52. Drive shaft 57 is coupled to an impeller 58. During operation, the rotation of drive shaft 57 and impeller 58 by the motor (not shown) disposed in motor section 51 causes blood to flow through blood pump 50. For example, blood may be drawn into one or more openings of inlet area 54, channeled through cannula 53, and expelled through one or more openings of outlet area 56. Alternatively, by, for example, reversing the direction of rotation of drive shaft 57 and impeller 58, blood may be drawn into one or more openings of outlet area 56, channeled through cannula 53, and expelled through one or more openings of inlet area 54.

In addition to optical fibers 28A and 28B, a power-supply line 59A and a purge-fluid line 59B extend through catheter 20. Electric power is provided to the motor (not shown) disposed in motor section 51 through power-supply line 59A. In some implementations, the electric power may be provided by controller 40. In some implementations, power-supply line 59A includes a plurality of electrical lines. A purge fluid (e.g., a dextrose solution) is delivered to the motor (not shown) disposed in motor section 51 through purge-fluid line 59B.

As shown in FIG. 3, sensor head 30 is positioned distally relative to sensor head 60, which is positioned more proximally. More specifically, sensor head 30 is attached to an external surface of cannula 53 by inlet area 54. As explained above, optical fiber 28A extends through tube 27, and sensor head 30 is located at the distal end of tube 27. Furthermore, sensor head 60 is attached to an external surface of pump section 52 by outlet area 56. Optical fiber 28B extends through a tube 21, which is attached to the external surface of blood pump 50 along to motor section 51 and pump section 52. Sensor head 60 is located at the distal end of tube 21. Tube 21 may be structured in much the same way as tube 27. For example, in some implementations, one or more additional optical fibers may extend through tube 21. As another example, in some implementations, tube 21 may encase all or some of the portion of fiber 28B that extends through catheter 20. As yet another example, in some implementations, tube 21 may be formed from a polymer (e.g., polyurethane) and/or a metal alloy (e.g., nitinol). Bulges or protuberances 35 and 65 are provided by sensor heads 30 and 60, respectively, to protect sensor heads 30 and 60 from colliding with, for example, a sluice valve or a hemostatic valve when blood pump 50 is being introduced into a patient’s vascular system. Additionally, as explained below, sensor heads 30 and 60 may be positioned within depressions in cannula 53 and pump section 52, respectively, to further protect them.

In other implementations, sensor heads 30 and/or 60 may be positioned at different locations. For example, sensor head 30 may be attached to an external surface of tip 55. In such implementations, tube 27 may be attached to the external surface of blood pump 50 along motor section 51, pump section 52, and/or cannula 53. As another example, sensor head 60 may be attached to an external surface of motor section 51. In such implementations, tube 21 may only be attached to the external surface of blood pump 50 along motor section 51, and not pump section 52. As yet another example, sensor heads 30 and/or 60 may be attached to an external surface of catheter 20. In such implementations, tubes 21 and/or 27 may be removed from blood pump 50. In other implementations, sensor heads 30 and/or 60 may be attached to an internal surface of blood pump 50. For example, sensor head 30 may be attached to an internal surface of cannula 53. As another example, sensor head 60 may be attached to an internal surface of pump section 52. Similarly, in other implementations, all or some of tubes 21 and/or 27 may be attached to an internal surface of blood pump 50.

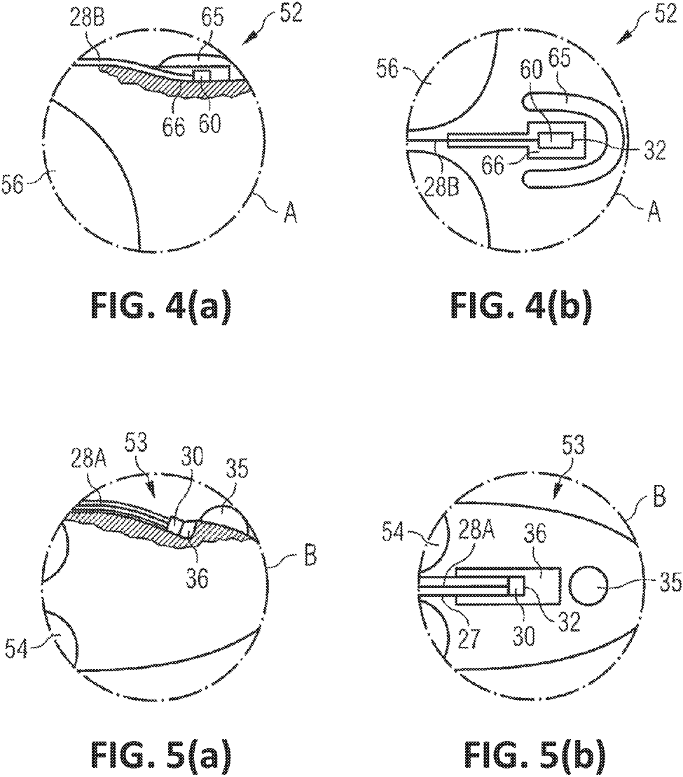

FIGS. 4A and 4B provide cross-sectional and top-down views, respectively, of detail A of FIG. 3. As shown, sensor head 60 is positioned in a depression 66 provided on an external surface of pump section 52. Furthermore, depression 66 is partially surrounded by a U-shaped bulge 65. In other implementations, bulge 65 may be replaced with an O-shaped bulge. In some implementations, bulge 65 may be formed by a bead of bonding agent. In some implementations, bulge 65 may be welded on or soldered onto the external surface of pump section 52. In some implementations, bulge 65 may form an integral part of pump section 52.

FIGS. 5A and 5B provide cross-sectional and top-down views, respectively, of detail B of FIG. 3. As shown, sensor head 30 is positioned in a depression 36 provided on an external surface of cannula 53. Furthermore, depression 36 is positioned adjacent to a point-shaped bulge 35. In other implementations, bulge 35 may be replaced with a U-shaped bulge or an O-shaped bulge. In some implementations, bulge 35 may be formed by a bead of bonding agent. In some implementations, bulge 35 may be welded on or soldered onto the external surface of cannula 53. In some implementations, bulge 35 may form an integral part of cannula 53.

Various modifications can be made to the ventricular support system of FIGS. 1 through 5(b) and one or more of its components. For example, the system can be modified to accommodate a variety of different intravascular blood pumps, such as the Impella 2.5®, Impella 5.0®, Impella 5.5®, Impella LD®, Impella RP®, and Impella CP® catheters from Abiomed, Inc., Danvers, MA. As another example, optical sensor heads 30 and/or 60 may be replaced with electrical pressure sensors (e.g., strain-gauge sensors). As yet another example, one or more sensors (e.g., optical and/or electrical sensors) may be added to blood pump 50. In such implementations, the one or more additional sensors may be used to measure, for example, the pressure within a patient’s femoral artery (e.g., when blood pump 50 is inserted percutaneously via the femoral artery) or axillary artery (e.g., when blood pump 50 is inserted percutaneously via the axillary artery). As yet another example, one or more components of the ventricular support system of FIGS. 1 through 5(b) may be separated or combined. For example, display 41 may be incorporated into another device in communication with controller 40 (e.g., wirelessly or through one or more electrical lines). As another example, the portions of optical fibers 28A and 28B extending through catheter 20 may be combined into a single optical fiber.

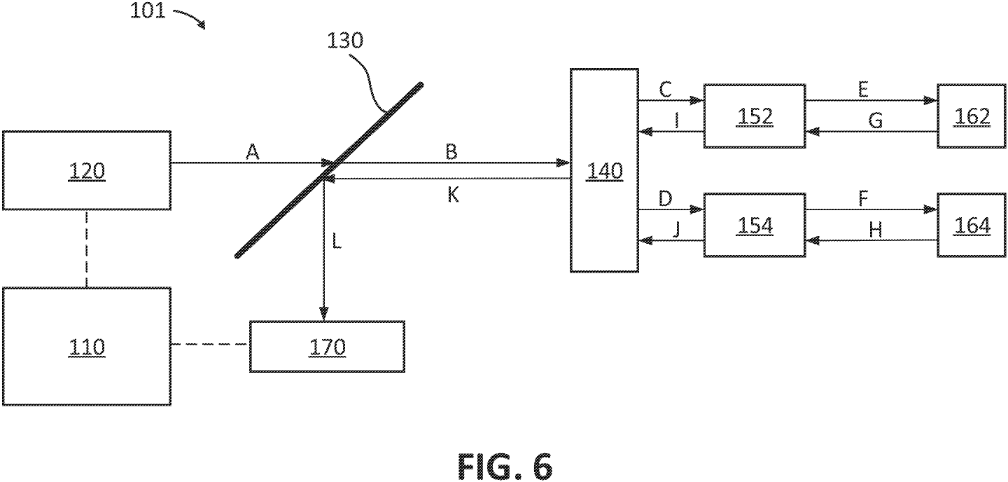

FIG. 6 illustrates a multi-sensor interferometry system 101. As shown, system 101 includes a controller 110, a light source 120, a beam splitter 130, a fiber optic splitter 140, filters 152 and 154, sensor heads 162 and 164, and a photodetector 170. However, system 101 may also include additional components, such as one or more collimating lenses and/or one or more focusing lenses. Controller 110 may include one or more processors, one or more application specific integrated circuits (ASICs), and/or other similar components. Controller 110 may also include a memory medium, such as a hard-drive, memory card, ROM, RAM, DVD, CD-ROM, write-capable, and/or read-only memory, that is capable of storing information. Light source 120 may include one or more incandescent lamps, one or more fluorescent lamps, one or more light-emitting diodes (LEDs), and/or one or more lasers. Light source 120 may, for example, produce white light (e.g., a distribution of wavelengths between approximately 400 nm and 700 nm) or one or more specific colors of light within the visible light spectrum (e.g., a combination of blue light and red light). Beam splitter 130 may, for example, be a plate beam splitter (e.g., a half-silvered mirror) or a cube beam splitter (e.g., a pair of triangular glass prisms that are glued together). Fiber optic splitter 140 may, for example, be a Fused Biconical Taper (FBT) splitter or a Planar Lightwave Circuit (PLC) splitter. Filters 152 and 154 may be short-pass filters, long-pass filters, or band-pass filters. Filters 152 and 154 may, for example, be absorptive filters (e.g., colored glass) or dichroic filters (e.g., coated glass). Sensor heads 162 and 164 may, for example, be structured much like sensor heads 30 and 60. Photodetector 170 may, for example, be a Charge Coupled Device (CCD) or Complementary Metal Oxide Semiconductor (CMOS) image sensor.

The components of system 101 may be directly or indirectly coupled together. For example, light source 120 and beam splitter 130 may be directly coupled together. Alternatively, light source 120 and beam splitter 130 may, for example, be coupled together through one or more optical fibers and/or one or more other optically transparent components. As another example, beam splitter 130 and fiber optic splitter 140 may be directly coupled together. Alternatively, beam splitter 130 and fiber optic splitter 140 may, for example, be coupled together through one or more optical fibers and/or one or more other optically transparent components. As yet another example, fiber optic splitter 140 and filters 152 and 154 may be directly coupled together. Alternatively, fiber optic splitter 140 and filters 152 and 154 may, for example, be coupled together through one or more optical fibers and/or one or more other optically transparent components. As yet another example, filters 152 and 154 may be directly coupled to sensor heads 162 and 164, respectively. Alternatively, filters 152 and 154 and sensor heads 162 and 164 may, for example, be coupled together through one or more optical fibers and/or one or more other optically transparent components. As yet another example, beam splitter 130 and photodetector 170 may be directly coupled together. Alternatively, beam splitter 130 and photodetector 170 may, for example, be coupled together through one or more optical fibers and/or one or more other optically transparent components.

The components of system 101 may form a Fabry-Perot interferometer. For example, each of sensor heads 162 and 164 may include a Fabry-Perot cavity having a pressure-sensitive membrane and a pair of partially reflective mirrors. During operation, controller 110 may control light source 120 to transmit a light beam (see arrow A) towards beam splitter 130. The light beam may be partially reflected by beam splitter 130. The portion of the light beam that passes through beam splitter 130 (see arrow B) may travel towards fiber optic splitter 140 and be split into two separate light beams (see arrows C and D) by fiber optic splitter 140. One of those light beams may travel through filter 152 towards sensor head 162 (see arrow E), and the other light beam may travel through filter 154 towards sensor head 164 (see arrow F). As the two light beams interact with the Fabry-Perot cavities of sensor heads 162 and 164, respectively, they may each be partially and multiply reflected by the pair of partially reflective mirrors to produce a plurality of interfering rays (see arrows G and H). The plurality of reflected, interfering rays may travel through filters 152 and 154 towards fiber optic splitter 140 (see arrows I and J) and be combined by fiber optic splitter 140 (see arrow K). The combined rays may travel towards beam splitter 130 and be partially reflected by beam splitter 130. The portion of the combined rays that is reflected by beam splitter 130 (see arrow L) may be received by photodetector 170. As the membranes of sensor heads 162 and 164 move, for example, in response to pressures acting on sensor heads 162 and 164, the interference pattern formed by the combined rays changes. Collectively, controller 110 and/or photodetector 170 may, for example, derive pressure measurements (e.g., ventricular pressure and/or aortic pressure) from these changing interference patterns.

Advantageously, system 101 only includes a single light source (i.e., light source 120) for transmitting a light beam to the sensor heads (i.e., sensor heads 162 and 164), and a single photodetector (i.e., photodetector 170) for receiving the plurality of interfering rays reflected from the sensor heads. Conventional ventricular support systems typically include separate interferometry systems for each sensor head. For example, a conventional implementation of the ventricular support system of FIGS. 1 through 5(b) would include two light sources and two photodetectors. However, by using the multi-sensor interferometry system of FIG. 6, only one light source and one photodetector is necessary. Moreover, by using the multi-sensor interferometry system of FIG. 6, only a single set of lenses (e.g., one or more collimating lenses and/or one or more focusing lenses) is necessary. As a result, system 101 can be used to improve the portability of the ventricular support system of FIGS. 1 through 5(b).

The above-noted advantages are achieved, at least in part, by dividing the spectrum of light produced by light source 120 into two different sub-spectrums that are provided to sensor heads 162 and 164. As explained above, a portion of the light beam produced by light source 120 travels through beam splitter 130 (see arrow B). Fiber optic splitter 140 divides that light beam into two separate light beams (see arrows C and D). In some implementations, fiber optic splitter 140 may be configured to provide approximately equal splitter ratios. In other implementations, fiber optic splitter 140 may be configured to provide unequal splitter ratios. Filters 152 and 154 filter the two light beams produced by fiber optic splitter 140. Furthermore, filters 152 and 154 permit different spectrums of light to pass through. For example, filter 152 may be a short-pass filter that permits wavelengths of light below a predetermined threshold (e.g., a value between 500 nm and 600 nm) to pass through, and filter 154 may be a long-pass filter that permits wavelengths of light above a predetermined threshold (e.g., a value between 500 nm and 600 nm) to pass through. As another example, filters 152 and 154 may be band-pass filters. For example, filter 152 may permit blue light to pass through, and filter 154 may permit red light to pass through. In some implementations, filters 152 and 154 may permit different overlapping spectrums of light to pass through. In other implementations, filters 152 and 154 may permit different non-overlapping spectrums of light to pass through.

As explained above, photodetector 170 receives a combination of the plurality of interfering rays reflected from sensor heads 162 and 164 (see arrow L). Controller 110 and/or photodetector 170 may digitally process the combined rays to separate the interference patterns created by sensor heads 162 and 164. For example, photodetector 170 may capture an image of the combined interference patterns, and controller 110 may digitally filter that image to separate the interference patterns created by sensor heads 162 and 164. For example, if filter 152 is a band-pass filter configured to permit blue light to pass through and if each pixel of the image has red, green, and blue values, controller 110 may set the red and green values of each pixel to zero to obtain the interference pattern created by sensor head 162. Similarly, if filter 154 is a band-pass filter configured to permit red light to pass through and if each pixel of the image has red, green, and blue values, controller 110 may set the green and blue values of each pixel to zero to obtain the interference pattern created by sensor head 164.

When combined with the ventricular support system of FIGS. 1 through 5(b), the components of system 101 may be positioned at a variety of different locations. For example, in some implementations, controller 40 of FIG. 1 may include controller 110, light source 120, beam splitter 130, fiber optic splitter 140, filters 152 and 154, and photodetector 170, and blood pump 50 of FIG. 1 may include sensor heads 162 and 164 (e.g., as sensor heads 30 and 60). In such implementations, the proximal ends of optical fibers 28A and 28B may be coupled to filters 152 and 154, respectively. As another example, in some implementations, controller 40 of FIG. 1 may include controller 110, light source 120, beam splitter 130, and photodetector 170, and blood pump 50 of FIG. 1 may include fiber optic splitter 140, filters 152 and 154, and sensor heads 162 and 164. In such implementations, a single optical fiber may extend through all or some of catheter 20. A distal end of the single optical fiber may be attached to fiber optic splitter 140. In such implementations, fiber optic splitter 140 and filters 152 and 154 may be positioned within the distal end of catheter 20 near point 22 (e.g., within 5 cm of point 22). Alternatively, fiber optic splitter 140 and filters 152 and 154 may be positioned on an internal or external surface of motor section 51, pump section 52, cannula 53, and/or tip 55. As yet another example, in some implementations, one or more separate devices (not shown), at least one of which is communicatively coupled to controller 40 of FIG. 1, may include controller 110, light source 120, beam splitter 130, fiber optic splitter 140, filters 152 and 154, and photodetector 170, and blood pump 50 of FIG. 1 may include sensor heads 162 and 164 (e.g., as sensor heads 30 and 60).

Various modifications can be made to system 101. For example, system 101 may include one or more additional sensor heads and corresponding filters. Furthermore, in such implementations, fiber optic splitter 140 may divide the portion of the light beam produced by light source 120 that travels through beam splitter 130 (see arrow B) into three or more separate light beams (rather than just two light beams). In such implementations, the corresponding filters may permit different overlapping or non-overlapping spectrums of light to pass through. For example, a first band-pass filter may be configured to permit blue light to pass through, a second band-pass filter may be configured to permit green light to pass through, and a third band-pass filter may be configured to permit red light to pass through. As more sensor heads are added to system 101 in this manner, the potential benefits (e.g., reduced size and/or power consumption) of incorporating system 101 into a ventricular support system increase.

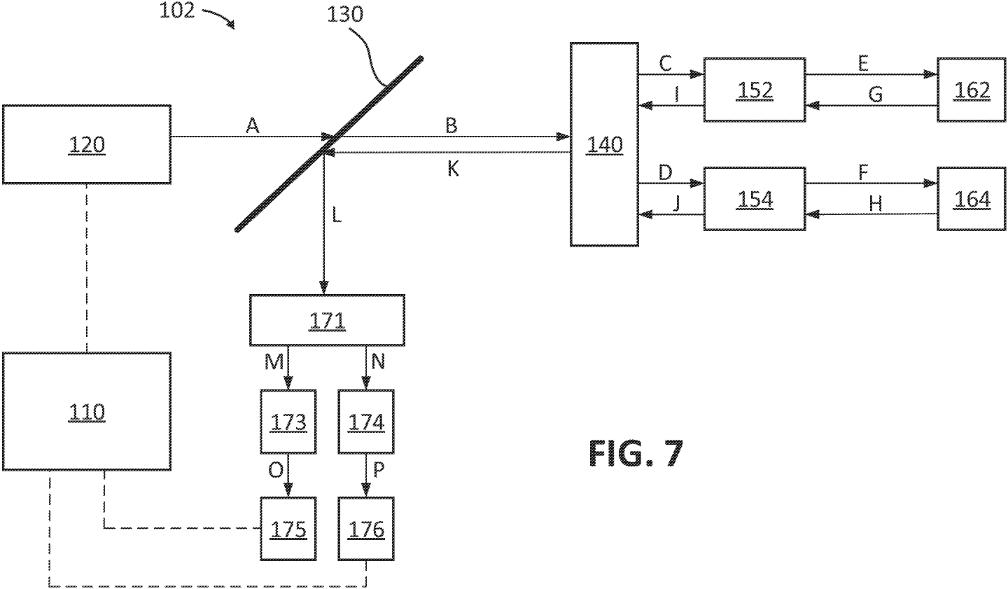

As another example, in some implementations, photodetector 170 may be replaced by a fiber optic splitter, two or more filters, and two or more separate photodetectors. For example, as shown in FIG. 7, a multi-sensor interferometry system 102 includes controller 110, light source 120, beam splitter 130, fiber optic splitter 140, filters 152 and 154, and sensor heads 166 and 168, as described above in relation to system 101 of FIG. 6. However, photodetector 170 has been replaced with fiber optic splitter 171, filters 173 and 174, and photodetectors 175 and 176. Fiber optic splitter 171 may, for example, be an FBT splitter or a PLC splitter. Filters 173 and 174 may be short-pass filters, long-pass filters, or band-pass filters. Filters 152 and 154 may, for example, be absorptive filters (e.g., colored glass) or dichroic filters (e.g., coated glass). Photodetectors 175 and 176 may, for example, be monochrome CCD or CMOS image sensors.

During operation, fiber optic splitter 171 receives a combination of the plurality of interfering rays reflected from sensor heads 162 and 164 (see arrow L) and divides them into two separate light beams (see arrows M and N). In some implementations, fiber optic splitter 171 may be configured to provide approximately equal splitter ratios. In other implementations, fiber optic splitter 171 may be configured to provide unequal splitter ratios. Filters 173 and 174 filter the two light beams produced by fiber optic splitter 171. Furthermore, filters 173 and 174 permit different spectrums of light to pass through. For example, filter 173 may be a short-pass filter that permits wavelengths of light below a predetermined threshold (e.g., a value between 500 nm and 600 nm) to pass through, and filter 174 may be a long-pass filter that permits wavelengths of light above a predetermined threshold (e.g., a value between 500 nm and 600 nm) to pass through. As another example, filters 173 and 174 may be band-pass filters. For example, filter 173 may permit blue light to pass through, and filter 174 may permit red light to pass through. In some implementations, filters 173 and 174 may permit different overlapping spectrums of light to pass through. In other implementations, filters 173 and 174 may permit different non-overlapping spectrums of light to pass through. Photodetectors 175 and 176 receive the filtered light beams produced by filters 173 and 174, respectively (see arrows O and P).

Advantageously, in relation to system 101, the configuration of system 102 may reduce some of the signal processing performed by controller 110. For example, in system 102, controller does not need to digitally process an image to obtain the separate the interference patterns created by sensor heads 162 and 164. Instead, controller 110 may, for example, receive an image of the interference pattern created by sensor head 162 from photodetector 175 and separately receive an image of the interference pattern created by sensor head 164 from photodetector 176. Furthermore, in some implementations, these images may have a higher resolution than the images described above in relation to FIG. 6. However, the additional components of system 102 may increase the cost, size and/or power consumption of system 102 in relation to system 101.

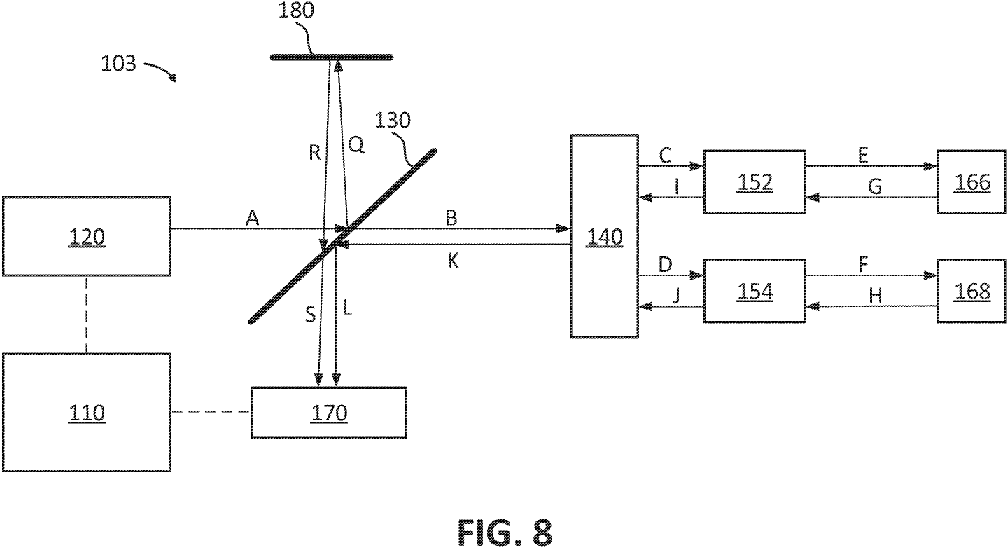

In some implementations, systems 101 and/or 102 may be modified to form a different type of interferometer, such as a Michelson interferometer or a Mach-Zehnder interferometer. For example, as shown in FIG. 8, a multi-sensor interferometry system 103 includes controller 110, light source 120, beam splitter 130, fiber optic splitter 140, filters 152 and 154, and photodetector 170, as described above in relation to system 101 of FIG. 6. However, system 103 also includes a mirror 180 to form a Michelson interferometer. Furthermore, sensor heads 162 and 164 have been replaced with sensor heads 166 and 168, respectively. Sensor heads 166 and 168 may, for example, be structured much like sensor heads 30 and 60. However, sensor heads 166 and 168 may include a single mirror attached to the bottom surface of a membrane, rather than a pair of partially reflective mirrors, as described above in relation to sensor heads 162 and 164.

During operation, controller 110 may control light source 120 to transmit a light beam (see arrow A) towards beam splitter 130. The light beam may be partially reflected by beam splitter 130. The portion of the light beam that is reflected by beam splitter 130 (see arrow Q) may travel towards mirror 180 and be reflected by mirror 180 back towards beam splitter 130 (see arrow R). A portion of the light beam reflected by mirror 180 may pass through beam splitter 130 and travel towards photodetector 170 (see arrow S). As explained above in relation to FIG. 6, the portion of the light beam produced by light source 120 that travels through beam splitter 130 (see arrow B) may travel towards fiber optic splitter 140 and be split into two separate light beams (see arrows C and D). After passing through filters 152 and 154, those light beams may be reflected by the mirrors of sensor heads 166 and 168 back towards filters 152 and 154 (see arrows G and H). The reflected signals may be combined by fiber optic splitter 140 (see arrow K), partially reflected by beam splitter 130 (see arrow L), and received by photodetector 170.

Collectively, controller 110 and/or photodetector 170 may digitally process the light beams received by photodetector 170 in much the same way described above in relation to FIG. 6. However, in the implementation of FIG. 8, photodetector 170 receives a reference light beam reflected by mirror 180 (see arrow S) and a combination of the light beams reflected by the mirrors of sensor heads 166 and 168 (see arrow L). Together, these light beams form a combined interference pattern. As the membranes of sensor heads 166 and 168 move, for example, in response to pressures acting on sensor heads 166 and 168, the interference pattern changes. Collectively, controller 110 and/or photodetector 170 may, for example, derive pressure measurements (e.g., ventricular pressure and/or aortic pressure) from these changing interference patterns.

From the foregoing and with reference to the various figure drawings, those skilled in the art will appreciate that certain modifications can also be made to the present disclosure without departing from the scope of the same. While several implementations of the disclosure have been shown in the drawings, it is not intended that the disclosure be limited thereto, as it is intended that the disclosure be as broad in scope as the art will allow and that the specification be read likewise. Therefore, the above description should not be construed as limiting, but merely as exemplifications of particular implementations. Those skilled in the art will envision other modifications within the scope and spirit of the claims appended hereto.

1. A ventricular support system comprising:
a light source configured to transmit light;
an intravascular blood pump comprising a first sensor head and a second sensor head wherein the first sensor head is positioned distally relative to the second sensor head;
a first filter directly or indirectly coupled to the first sensor head, wherein the first filter permits a first sub-spectrum of the light transmitted by the light source to pass through it;
a second filter directly or indirectly coupled to the second sensor head, wherein the second filter permits a second sub-spectrum of the light transmitted by the light source to pass through it, and wherein the first and second sub-spectrums are different;
a first fiber optic splitter directly or indirectly coupled to the first and second filters, wherein the first fiber optic splitter is configured to split at least some of the light transmitted by the light source such that a first portion is transmitted to the first filter and a second portion is transmitted to the second filter, and wherein the first fiber optic splitter is configured to combine a plurality of light beams reflected from the first and second sensor heads; and
a first photodetector configured to receive at least some of the combined plurality of light beams reflected from the first and second sensor heads.

2. The system of claim 1, wherein the first filter is directly coupled to the first sensor, and wherein the second filter is directly coupled to the second sensor.

3. The system of claim 2, wherein the first fiber optic splitter is coupled to the first filter through a first optical fiber extending through at least a portion of a catheter of the intravascular blood pump, and wherein the first fiber optic splitter is coupled to the second filter through a second optical fiber extending through at least a portion of the catheter.

4. The system of claim 1, wherein the first filter is coupled to the first sensor head through a first optical fiber extending through at least a portion of a catheter of the intravascular blood pump, and wherein the second filter is coupled to the second sensor head through a second optical fiber extending through at least a portion of the catheter.

5. The system of claim 1, further comprising:
a beam splitter coupled to the first fiber optic splitter through a first optical fiber extending through at least a portion of a catheter of the intravascular blood pump.

6. The system of claim 1, wherein each of the first and second sensor heads comprise a cavity and a pressure-sensitive membrane.

7. The system of claim 6, wherein the cavity and the pressure-sensitive membrane of each of the first and second sensor heads form part of a Fabry-Perot cavity.

8. The system of claim 6, further comprising:
a mirror configured to reflect at least some of the light transmitted by the light source towards the first photodetector,
wherein each of the first and second sensor heads comprises a mirror attached to a bottom surface of the pressure-sensitive membrane that faces the cavity.

9. The system of claim 1, wherein the first filter is a short-pass filter, and wherein the second filter is a long pass filter.

10. The system of claim 1, wherein the first and second filters are band-pass filters.

11. The system of claim 1, wherein the first and second sub-spectrums overlap.

12. The system of claim 1, wherein the first and second sub-spectrums do not overlap.

13. The system of claim 1, further comprising:
a third filter directly or indirectly coupled to a third sensor head of the intravascular blood pump, wherein the third filter permits a third sub-spectrum of the light transmitted by the light source to pass through it, and wherein the first, second, and third sub-spectrums are different,
wherein the first fiber optic splitter is configured to split at least some of the light transmitted by the light source such that a third portion is transmitted to the third filter, and wherein the first fiber optic splitter is configured to combine a plurality of light beams reflected from the first, second, and third sensor heads.

14. The system of claim 1, further comprising:
a controller communicatively coupled to the light source and the first photodetector.

15. The system of claim 14, wherein the first photodetector is configured to capture an image of a combined interference pattern formed by the combined plurality of light beams reflected from the first and second sensor heads, and wherein the controller is configured to digitally filter the image to separate the interference patterns created by the first and second sensor heads.

16. The system of claim 1, further comprising:
a second photodetector configured to receive at least some of the combined plurality of light beams reflected from the first and second sensor heads;
a third filter directly or indirectly coupled to the first photodetector, wherein the third filter permits the first sub-spectrum of the light transmitted by the light source to pass through it;
a fourth filter directly or indirectly coupled to the second photodetector, wherein the fourth filter permits the second sub-spectrum of the light transmitted by the light source to pass through it; and
a second fiber optic splitter directly or indirectly coupled to the third and fourth filters, wherein the second fiber optic splitter is configured to split at least some of the combined plurality of light beams reflected from the first and second sensor heads such that a first portion is transmitted to the third filter and a second portion is transmitted to the fourth filter.

17. A method comprising:
transmitting, from a light source, a first light beam;
splitting, with a fiber optic splitter, at least some of the first light beam received by the fiber optic splitter into a second light beam and a third light beam;
filtering, with a first filter, at least some of the second light beam received by the first filter, wherein the first filter permits a first sub-spectrum of the second light beam to pass through it;
filtering, with a second filter, at least some of the third light beam received by the second filter, wherein the second filter permits a second sub-spectrum of the third light beam to pass through it, and wherein the first and second sub-spectrums are different;
reflecting, with a first sensor head of an intravascular blood pump, at least some of the filtered second light beam to produce a first reflected light beam;
reflecting, with a second sensor head of the intravascular blood pump, at least some of the filtered third light beam to produce a second reflected light beam;
combining, with the fiber optic splitter, the first and second reflected light beams; and
receiving, with a photodetector, at least some of the combined first and second reflected light beams.

18. The method of claim 17, further comprising:
deriving a ventricular pressure and an aortic pressure from the at least some of the combined first and second reflected light beams received by the photodetector.

19. The method of claim 17, wherein each of the first and second sensor heads comprises a cavity and a pressure-sensitive membrane.

20. The method of claim 19, wherein the cavity and the pressure-sensitive membrane of each of the first and second sensor heads form part of a Fabry-Perot cavity.
